SEQUENCE LISTING (17 sequences)

SEQ ID NO: 1 - protein, 288 aa, Tetrahymena thermophila
MKLQLLLLVCLSFAACQSFTYTQSLAQDLAGFSLASYCNPKSIEQWNCGCACDKNPQGLRNVTILFNSTLQASGYLGYSTHHDAIVVVFRGTVPWLIENWIADLNTFKTQYPLCQNCYVHQGFYNQFKQLKSQLVTSFTSLRQLYPNAKVFVTGHSLGAAMSAHSIPVIYQLNGNKPIDAFYNYGCPRVGDQTYANWFNSQNFALEYGRINNAADPVPHLPPLLYPFSFFHYNHEIFYPSFVLFGNQHNQCQNAETIFGADGVIIAANVLDHLTYFGWDWSGSILTCQ

SEQ ID NO: 2 - protein, 288 aa, Tetrahymena thermophila
MKSIFLLIISLLLASCSQFQYNETLAQDLAGFSLASYCNPKYLQQWNCGSACKKNPNGLTDFSYLYNKTLKASGYIGYSAHHDAIIVVFRGTVPWLIQNWIADLNTIKIQYPFCENCYVHKGFYKQFNQLKSQLIQSFTEIRQKYPSSKIFVTGHSLGAAMSFHSMPIIFELNGNKPIDAFYNYGSPRVGNEAYATWFNLQNFALQYGRINNAADPVPHLPPILFPFQFYHTNHEIFYTSFIEDGNKYEQCLDAEHKLCANSKIIAASVRDHLSYFGWNWATSILTCQ

SEQ ID NO: 3 - protein, 308 aa, Tetrahymena thermophila
MNKLQVLFIAAIVCTIGSTVYLLNKSSSDVQESQLTFPYDENLAENLAGFSMASYCKASKIENWNCGASCKKNPEGLQDVYIMKNKTMNAAGFLAYSPAHDAIVVVFRGTVPWLIKNWISDINTVKTKYSRCEKCYVHLGFFNAFKELQDQILTEFPKLKAKYPYSKNINYSKLNRRHSLGAAMSTHAVPVIYELNGNKPIDAFYNFGSPRVGDENYHQWFDSQNFTLQYGRINHRADPVPHLPPNYSPFTFTHIDHEVFYQTFKKPYTQCIETESLECADGIKIPLDIPDHLSYFGWDWATDILACQ

SEQ ID NO: 4 - protein, 486 aa, Tetrahymena thermophila
MSKLQFVLIAALLLVAVSAKTAQEWKTRNIYQIITDRFALGDGSKPYCDLNKQPYYCGGNFKGIENNLDYIQGMGFNAIWISPVPDNYPNQFHGYAAKNMTEINSHFGGADGLKSLIDACHKRDIWVMIDVVANHMGNTDQNYSENIPFNSPDHYHSYCIISDSDFATKNMYNIQHCRLAGLADLNQENSYVSNYLVNWIKDLVQTYNVDGIRIDTVPEVHPDFWKQYTTSAGVFATGEVFDGDFGYVASYIPSVGSVLNYPWYFNMRDIFVNQKDMWGVRTYYQNWANQHADLSILTPFVDNHDNPRFLSDQVFNNGKDRNTRIQLLKGYTTFTLTSIGIPILYYGTEQYFSGNTDPYDREPLWNSLNTNSDMYKYVQSILYAKNITNAGLQGQTEAYCDQDLYAFFRGKLFVGVTSKFNQVTRQIVTHPFSNGEVICNIFYPTQDCLTITNNSFNLVLIGGEAKIFVPKSLLQKSSQFLRSASS

SEQ ID NO: 5 - protein, 324 aa, Tetrahymena thermophila
MSQKITVTLVAIAAIAAITAAGIYYQNHQASQLEKSFKRNTILEQWNEFKQKFGKKYADQEFERYRIGVFAQNLEVIKNDPSFGVTKFMDMTPQEFEQSYLSLQLQQNFNAEKVDGDFNGDIDWTQKGAVTPVKDQGSCGSCWAFSAIGAVESALILNGEDKNINLAEQELVDCATTPKYENEGCNGGWMDSAFDYIIDEKISQTKDYKYTARDGKCKDTSSFEKKSISGYKDIPQGDCKSLLNALSQQPVAIAVDASSWQFYNKGVLSSCGSRLNHGVLLTGYVNETYKVKNSWGTSWGEKGFIQLKSGNSCGLCNAASYPLA

SEQ ID NO: 6 - protein, 320 aa, Tetrahymena thermophila
MNKTLILALVGVLALTATTLVAFHNHSHNIRVDQDPATLFKQFKQTYNKKYADPTFETYRFGVFTQNLEIVKTDSTFGVTQFMDLTPAEFAQQFLTLHEKVNSTEVYRAQGEATEVDWTAKGKVTPVKNQGSCGSCWAFSTIGAVESALLIAGQGEQNTLNLAEQELVDCAKSPKYDSEGCNGGWMVEGFKYIIDNKISQTANYPYTAKDGKCKDTSSFKKFSISKYAEIPQGDCNSLNSALEQGPISVAVDATNFQFYTSGVFKNCKANLNHGVLLVANVDSSLKIKNSWGPSWGEKGFIRLAAGNTCGVCNAASYPIV

SEQ ID NO: 7 - protein, 325 aa, Tetrahymena thermophila
MRTQLLIAAALGLTLLGLTSYLFLHKSTQVGYTDDQINMWKGFKKTYNKKFSSEDADQEAYRMNVFFDNVEYASQDSTMGITKFMDLTPVEFAQLYLNPIENVEGSIETFQAIQANGDIVVDWVAKGAVTPVKDQGGCGGCWSFATTGGVEGANFVYKNVLPNLSEQQLIDCDTQNSGCGGGLRDVALNYVKATGLATEQDYPYEAKDGKCRLEGKSHPWTVSGYTSIKQCADLVTAIQKAPVTVGIDASNLQFYTGGIFSKCATNINHGVLLVGYDSVNQSWKVKNSWGPNFGEHGYFQLSAKVTGDQIANTCGICSRAYAPYI

SEQ ID NO: 8 - protein, 272 aa, Tetrahymena thermophila
QSFTYTQSLAQDLAGFSLASYCNPKSIEQWNCGCACDKNPQGLRNVTILFNSTLQASGYLGYSTHHDAIVVVFRGTVPWLIENWIADLNTFKTQYPLCQNCYVHQGFYNQFKQLKSQLVTSFTSLRQLYPNAKVFVTGHSLGAAMSAHSIPVIYQLNGNKPIDAFYNYGCPRVGDQTYANWFNSQNFALEYGRINNAADPVPHLPPLLYPFSFFHYNHEIFYPSFVLFGNQHNQCQNAETIFGADGVIIAANVLDHLTYFGWDWSGSILTCQ

SEQ ID NO: 9 - protein, 270 aa, Tetrahymena thermophila
FQYNETLAQDLAGFSLASYCNPKYLQQWNCGSACKKNPNGLTDFSYLYNKTLKASGYIGYSAHHDAIIVVFRGTVPWLIQNWIADLNTIKIQYPFCENCYVHKGFYKQFNQLKSQLIQSFTEIRQKYPSSKIFVTGHSLGAAMSFHSMPIIFELNGNKPIDAFYNYGSPRVGNEAYATWFNLQNFALQYGRINNAADPVPHLPPILFPFQFYHTNHEIFYTSFIEDGNKYEQCLDAEHKLCANSKIIAASVRDHLSYFGWNWATSILTCQ

SEQ ID NO: 10 - protein, 289 aa, Tetrahymena thermophila
VYLLNKSSSDVQESQLTFPYDENLAENLAGFSMASYCKASKIENWNCGASCKKNPEGLQDVYIMKNKTMNAAGFLAYSPAHDAIVVVFRGTVPWLIKNWISDINTVKTKYSRCEKCYVHLGFFNAFKELQDQILTEFPKLKAKYPYSKNINYSKLNRRHSLGAAMSTHAVPVIYELNGNKPIDAFYNFGSPRVGDENYHQWFDSQNFTLQYGRINHRADPVPHLPPNYSPFTFTHIDHEVFYQTFKKPYTQCIETESLECADGIKIPLDIPDHLSYFGWDWATDILACQ

SEQ ID NO: 11 - protein, 467 aa, Tetrahymena thermophila
KTAQEWKTRNIYQIITDRFALGDGSKPYCDLNKQPYYCGGNFKGIENNLDYIQGMGFNAIWISPVPDNYPNQFHGYAAKNMTEINSHFGGADGLKSLIDACHKRDIWVMIDVVANHMGNTDQNYSENIPFNSPDHYHSYCIISDSDFATKNMYNIQHCRLAGLADLNQENSYVSNYLVNWIKDLVQTYNVDGIRIDTVPEVHPDFWKQYTTSAGVFATGEVFDGDFGYVASYIPSVGSVLNYPWYFNMRDIFVNQKDMWGVRTYYQNWANQHADLSILTPFVDNHDNPRFLSDQVFNNGKDRNTRIQLLKGYTTFTLTSIGIPILYYGTEQYFSGNTDPYDREPLWNSLNTNSDMYKYVQSILYAKNITNAGLQGQTEAYCDQDLYAFFRGKLFVGVTSKFNQVTRQIVTHPFSNGEVICNIFYPTQDCLTITNNSFNLVLIGGEAKIFVPKSLLQKSSQFLRSASS

SEQ ID NO: 12 - protein, 303 aa, Tetrahymena thermophila
GIYYQNHQASQLEKSFKRNTILEQWNEFKQKFGKKYADQEFERYRIGVFAQNLEVIKNDPSFGVTKFMDMTPQEFEQSYLSLQLQQNFNAEKVDGDFNGDIDWTQKGAVTPVKDQGSCGSCWAFSAIGAVESALILNGEDKNINLAEQELVDCATTPKYENEGCNGGWMDSAFDYIIDEKISQTKDYKYTARDGKCKDTSSFEKKSISGYKDIPQGDCKSLLNALSQQPVAIAVDASSWQFYNKGVLSSCGSRLNHGVLLTGYVNETYKVKNSWGTSWGEKGFIQLKSGNSCGLCNAASYPLA

SEQ ID NO: 13 - protein, 298 aa, Tetrahymena thermophila
FHNHSHNIRVDQDPATLFKQFKQTYNKKYADPTFETYRFGVFTQNLEIVKTDSTFGVTQFMDLTPAEFAQQFLTLHEKVNSTEVYRAQGEATEVDWTAKGKVTPVKNQGSCGSCWAFSTIGAVESALLIAGQGEQNTLNLAEQELVDCAKSPKYDSEGCNGGWMVEGFKYIIDNKISQTANYPYTAKDGKCKDTSSFKKFSISKYAEIPQGDCNSLNSALEQGPISVAVDATNFQFYTSGVFKNCKANLNHGVLLVANVDSSLKIKNSWGPSWGEKGFIRLAAGNTCGVCNAASYPIV

SEQ ID NO: 14 - protein, 301 aa, Tetrahymena thermophila
HKSTQVGYTDDQINMWKGFKKTYNKKFSSEDADQEAYRMNVFFDNVEYASQDSTMGITKFMDLTPVEFAQLYLNPIENVEGSIETFQAIQANGDIVVDWVAKGAVTPVKDQGGCGGCWSFATTGGVEGANFVYKNVLPNLSEQQLIDCDTQNSGCGGGLRDVALNYVKATGLATEQDYPYEAKDGKCRLEGKSHPWTVSGYTSIKQCADLVTAIQKAPVTVGIDASNLQFYTGGIFSKCATNINHGVLLVGYDSVNQSWKVKNSWGPNFGEHGYFQLSAKVTGDQIANTCGICSRAYAPYI

SEQ ID NO: 15 - protein, 207 aa, Tetrahymena thermophila
FNGDIDWTQKGAVTPVKDQGSCGSCWAFSAIGAVESALILNGEDKNINLAEQELVDCATTPKYENEGCNGGWMDSAFDYIIDEKISQTKDYKYTARDGKCKDTSSFEKKSISGYKDIPQGDCKSLLNALSQQPVAIAVDASSWQFYNKGVLSSCGSRLNHGVLLTGYVNETYKVKNSWGTSWGEKGFIQLKSGNSCGLCNAASYPLA

SEQ ID NO: 16 - protein, 209 aa, Tetrahymena thermophila
EATEVDWTAKGKVTPVKNQGSCGSCWAFSTIGAVESALLIAGQGEQNTLNLAEQELVDCAKSPKYDSEGCNGGWMVEGFKYIIDNKISQTANYPYTAKDGKCKDTSSFKKFSISKYAEIPQGDCNSLNSALEQGPISVAVDATNFQFYTSGVFKNCKANLNHGVLLVANVDSSLKIKNSWGPSWGEKGFIRLAAGNTCGVCNAASYPIV

SEQ ID NO: 17 - protein, 209 aa, Tetrahymena thermophila
GDIVVDWVAKGAVTPVKDQGGCGGCWSFATTGGVEGANFVYKNVLPNLSEQQLIDCDTQNSGCGGGLRDVALNYVKATGLATEQDYPYEAKDGKCRLEGKSHPWTVSGYTSIKQCADLVTAIQKAPVTVGIDASNLQFYTGGIFSKCATNINHGVLLVGYDSVNQSWKVKNSWGPNFGEHGYFQLSAKVTGDQIANTCGICSRAYAPYI

(19) United States
(12) Patent Application Publication
(10) Pub. No.: US 2024/0191275 A1
(43) Pub. Date: Jun. 13, 2024

(54) PURIFICATION OF PROTEINS

(71) Applicant: CILIAN AG, Münster (DE)

(72) Inventors: Marcus Wolf William HARTMANN, Münster (DE); Ingo ALDAG, Münster (DE); Jan ROSSDORF, Rhede (DE); Stefan UTHOFF, Münster (DE); Florian SCHUMACHER, Steinfurt (DE)

(21) Appl. No.: 18/554,513

(22) PCT Filed: Apr. 11, 2022
(86) PCT No.: PCT/EP2022/059587

§ 371 Date: Oct. 9, 2023

(30) Foreign Application Priority Data
Apr. 9, 2021 (EP) 21167712.5

(51) Int. Cl. C12P 21/00; C12N 9/20; C12N 9/28

(52) U.S. Cl. CPC C12P 21/005; C12N 9/20; C12N 9/2417; C12Y 301/01003; C12Y 302/01001

(57) ABSTRACT

The present invention relates to a method for purifying proteins, which proteins are produced by homologous or heterologous expression in a ciliate host, from harvested cell culture fluid (HCCF) which was not chromatographically purified. The method comprises the steps of incubating a harvested cell culture fluid with a kosmotropic agent, efflorescing protein by formation of crystals, and, optionally, harvesting effloresced protein.

### FIELD OF THE INVENTION

The present application relates to the purification of proteins.

### BACKGROUND OF THE INVENTION

Proteins that are produced in biological systems require purification before they can be administered to patients. Different approaches exist for this purpose, each of which comes with different advantages and disadvantages. Oftentimes, increasing purity results in higher costs and decreased throughput, and vice versa.

Further, depending on the method of production, and the route of administration, different requirements exist with regard to the degree of purity. Proteins for external use need to be less purified than proteins for intravenous administration.

Generally, in the homologous or heterologous expression of proteins, irrespective of which host is chosen (Bacteria, yeasts, mammalian cells) the cell culture supernatant comprises, next to the components of the pre-harvest cell culture fluid and the protein to be produced, unwanted components like endotoxins, antibiotics, debris, catabolites, and the like.

One standard method for protein purification is chromatography. However, mass transfer limitations causes the necessity to design large chromatography columns which correspondingly require large amounts of resin. The resins have a limited life-period and, therefore, represent consumables. In addition, the resins are costly which is in particular the case for affinity chromatography resins or immobilized metal chromatography resins.

Generally, protein purification using a chromatographic purification step like chromatography is a costly endeavor, because it requires sophisticated equipment and consumables, and large amounts of expensive buffer solutions. Further, the throughput of chromatographic purification steps is often limited, demanding parallel processing to achieve sufficient yield, which in turn drives costs.

Still, for proteins used in human consumption or therapy, chromatography is still the method of choice, to satisfy the high demand regarding purity of the product.

However, therapy applications exist where large amounts of protein are needed. This applies, inter alia, for some enzyme replacement therapies, like pancreatic enzyme replacement therapy (PERT), but, inter alia, also for other oral enzyme replacement therapies. Here, chromatographic purification would drive costs beyond acceptable limits.

On the other hand, protein expression systems exist which offer relatively low production cost per gram of produced enzyme, like e.g. Ciliate expression systems. Chromatographic purification of the produced enzymes would affect the economic advantages these systems can offer.

It is hence one object of the present invention to provide a purification method that overcomes the above disadvantages.

This and further objects are met with methods and means according to the independent claims of the present invention. The dependent claims are related to specific embodiments.

### SUMMARY OF THE INVENTION

The present invention provides modified lipase enzymes. The invention and general advantages of its features will be discussed in detail below.

### BRIEF DESCRIPTION OF THE FIGURES

FIG. 1 shows an overview of N-glycosylation patterns of different taxa. Generally, the term “N-glycosylation” refers to glycosylation of the amino acid residue asparagine (N). Here, an oligosaccharide chain is attached by oligosaccharyltransferase to those asparagine residues which occur in the tripeptide sequences Asn-X-Ser or Asn-X-Thr, where X can be any amino acid except Pro. It is obvious that, while prokaryotes have no N-glycosylation at all, ciliates feature N-glycosylation patterns which very are simple and small, compared to other taxa, like mammals, yeast or transgenic plants.

FIG. 2 shows a photomicrograph (40×) of lipase crystals formed after incubation of harvested cell culture fluid (HCCF) which was not chromatographically purified, after incubation with 1000 mM ammonium sulfate for 130 mins.

FIG. 3 shows a photomicrograph (40×) of lipase crystals formed after incubation of harvested cell culture fluid (HCCF) which was not chromatographically purified, after incubation with 850 mM ammonium sulfate for 130 mins.

FIG. 4 shows a photomicrograph (40×) of lipase crystals formed after incubation of harvested cell culture fluid (HCCF) which was not chromatographically purified, after incubation with 700 mM ammonium sulfate for 130 mins.

FIG. 5 shows a photomicrograph (40×) of harvested cell culture fluid (HCCF) which was not chromatographically purified, after incubation with 3000 mM sodium chloride for 130 mins.

FIG. 6 shows a photomicrograph (10×) of harvested cell culture fluid (HCCF) which was not chromatographically purified, after incubation with 300 mM ammonium sulfate for 3 hrs.

FIG. 7 shows a photomicrograph (10×) of harvested cell culture fluid (HCCF) which was not chromatographically purified, after incubation with 300 mM ammonium sulfate for 20 hrs.

FIG. 8 shows results of a Rhodamine-Triglyceride-Agarose assay to determine the lipolytic activity of the supernatant and the crystals, after exposure of harvested cell culture fluid comprising lipase to different crystallization conditions.

FIG. 9 shows results of a BCA assay, in order to determine the total concentration of protein in the supernatant and the crystals, after exposure of harvested cell culture fluid to different crystallization conditions

FIG. 10 shows the protein crystallization scheme. The steps, which have been carried out during the crystallization process, are schematically depicted. Lipase enzyme was produced as discussed elsewhere herein in a ciliate expression system. Protein crystallization was initiated with 0.3 M ammonium sulphate. The increase of ammonium sulphate to 0.7 M was performed after 20 h of incubation at room temperature. The protein crystals have subsequently been washed two times and the supernatants have been discarded. The remaining wet lipase crystals were stored at −20° C.

FIGS. 11 and 12 Crystals of a-amylase from T. thermophila obtained after 93 h with 6% (w/v) PEG 4000 as the crystallization agent. Magnification: FIG. 11: 100×; FIG. 12: 400×; Bars indicate approx. 100 μm.

FIG. 13 shows results of a Rhodamine-Triglyceride assay to determine the lipolytic activity of the supernatant and the crystals, after exposure of harvested cell culture fluid comprising lipase to different crystallization conditions with NaH₂PO₄ as crystallization agent.

FIG. 14 shows Crystals of lipase 0120 from T. thermophila obtained after 115 h with 1.25 M NaH₂PO₄ final concentration as the crystallization agent. Bar indicates approx. 100 μm.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions.

According to a first aspect of the invention, a method for purifying proteins is provided, which proteins are produced by homologous or heterologous expression in a ciliate host, from harvested cell culture fluid (HCCF) which was not chromatographically purified.

The method comprises the steps of
a) incubating the harvested cell culture fluid with a kosmotropic agent,
b) efflorescing protein by formation of crystals, and
c) optionally, harvesting effloresced protein.

The method does not comprise a step of crosslinking the proteins before or after efflorescing.

The inventors have shown that such method, although not including a step of chromatographic purification nor a step of crosslinking of the proteins, provides excellent results with regard to purity and yield, as e.g. compared to a chromatographic purification, and offers, furthermore, substantial cost benefits as well.

The term “ciliate host”, as used herein, shall refer to the scientific phylum of Ciliophora, which are unicellular eukaryotes (“protozoa” or “protists”) characterized, among others, by their relatively large size (some species have up to 2 mm in length), their ciliated cell surface and by two different sorts of nuclei, i.e., a small, diploid micronucleus, and a large, polyploid macronucleus (used for protein expression). The latter is generated from the micronucleus by amplification of the genome and heavy editing. Ciliates, in particular Tetrahymena thermophila, have been used as expression hosts for the production of heterologous or homologous protein expression, as e.g. described in Weide et al. 2006, as well as in EP1350838 (U.S. Pat. No. 6,962,800), the content of which is incorporated herein by reference for enablement purposes.

As used herein, the term “pre-harvest cell culture fluid (CCF)” relates to a liquid medium which contains the ciliate host cells, and the culture media comprising inter alia the enzymes produced by homologous or heterologous expression.

As used herein, the term “harvested cell culture fluid (HCCF)” relates to a liquid medium which is obtained from pre-harvest cell culture fluid (CCF) by centrifugation, filtration, and/or similar separation methods.

As used herein, the term “not chromatographically purified” shall mean that the harvested cell culture fluid did not undergo purification based on chromatography. This does not exclude mere size-exclusion purification steps, like filtration, or density based purification steps, like centrifugation.

As used herein, the term “efflorescing” relates to a process in which protein is precipitated by formation of crystals. The terms “efflorescing” and “precipitating by formation of crystals” can be used interchangeably. Originally, the term “efflorescence” relates to a crystalline deposit that can form when water is present in or on brick, concrete, stone, stucco or other building surfaces. However, in the present context, its meaning relates to the process as outlined above.

An “effloresced protein”, as used herein, is a protein that has been obtained by such process of precipitating and formation of crystals. This means that an effloresced protein is a protein that is present in the form of crystals, as obtained according to the above method. In that sense, the terms “effloresced protein” and “crystallized protein” are being used interchangeably herein.

As used herein, the term “kosmotropic agent” relates to an agent which contributes to the stability and structure of water-water interactions in solutions. Kosmotropes cause water molecules to favorably interact, which stabilizes intramolecular interactions in macromolecules such as proteins, contrary to chaotropic salts, which have the opposite effect, in that they disrupt water structure, increase the solubility of nonpolar solvent particles, and destabilize solute aggregates.

Ionic kosmotropes tend to be small or have high charge density. Some ionic kosmotropes are CO₃²⁻, SO₄²⁻, HPO₄²⁻, Mg²⁺, Lit, Zn²⁺ and Al³⁺. A scale can be established if one refers to the Hofmeister series or looks up the free energy of hydrogen bonding (ΔG_{HB}) of the salts, which quantifies the extent of hydrogen bonding of an ion in water. For example, the kosmotropes CO₃²⁻, and OH⁻ have ΔG_{HB} between 0.1 and 0.4 J/mol, whereas the chaotrope SCN⁻ has a ΔG_{HB} between −1.1 and −0.9.

Other suitable kosmotropes include nonionic kosmotropes, which have no net charge but are very soluble and become very hydrated. Such kosmotropes encompass carbohydrates such as trehalose and glucose, polyalcohols, as well as proline and tert-butanol.

The present inventors have surprisingly shown that enzymes produced in ciliate hosts either by homologous or heterologous expression can simply and easily be purified by efflorescing and crystal formation after treatment with a kosmotrope, without the need of prior chromatographic purification.

This is quite surprising, because in the prior art literature, before being able to crystallize a protein, in particular a lipase, the protein always underwent prior purification step by chromatographic purification or similar. See, in this regard e.g., Hekmat (2015), who discusses the large-scale crystallization of proteins for purification and formulation.

Yet, though the author realizes that the crystallization can replace one or more chromatography steps in the process of protein purification, all proteins that are being investigated and eventually crystallized are purified ones, i.e., have undergone a chromatographic purification step before they are used.

Further, the present inventors have surprisingly shown that enzymes thus produced do not have to be crosslinked, as e.g. suggested by several prior art references.

U.S. Pat. No. 6,359,118B2 for example discloses the production and use of carbohydrate crosslinked glycoprotein crystals. The crosslinking is preferably accomplished by using a diamine crosslinking agent, like, e.g., hexamethylenediamine, diaminooctane, or ethylenediamine. The glycoprotein is preferably a lipase, more preferably a Candida rugosa lipase. Such crosslinked glycoprotein crystals are said to
display better stability to harsh environmental conditions,
while maintaining the structural and functional integrity of the glycoprotein backbone
   while the crosslinking method is said to provide that the concentration of glycoprotein is close to the theoretical packing limit that can be achieved for molecules of a given size, greatly yield achievable even in concentrated solutions.

Without being bound to theory, one reason for the fact that the method according to the invention delivers outstanding results without prior crosslinking of enzymes may lie in the fact that by using appropriate promoters, proteins, including homologous and heterologous enzymes, and in particular lipases, can be expressed in Ciliates in large quantities. In such way, high yields are provided which may make the crosslinking step (which is a step to increase the yield after efflorescing) obsolete.

Further, without being bound to theory, one reasons for the fact that the method according to the invention delivers outstanding results without prior chromatographic purification of enzymes may lie in the fact that ciliates are capable to secrete large amounts of protein into the cell free supernatant, so that a crystallization is possible even without prior chromatography and/or crosslinking.

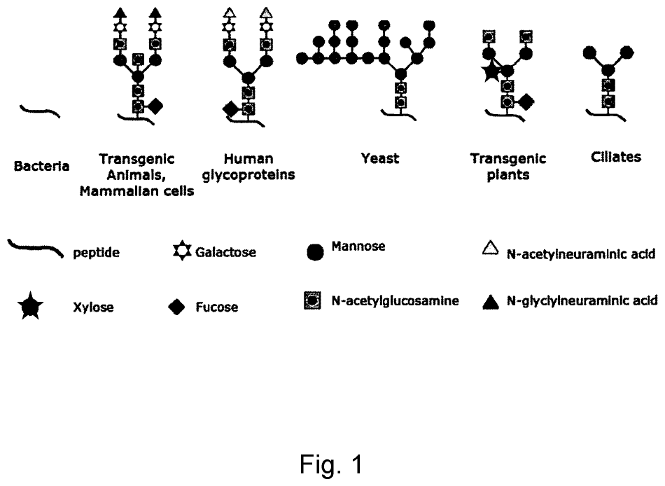

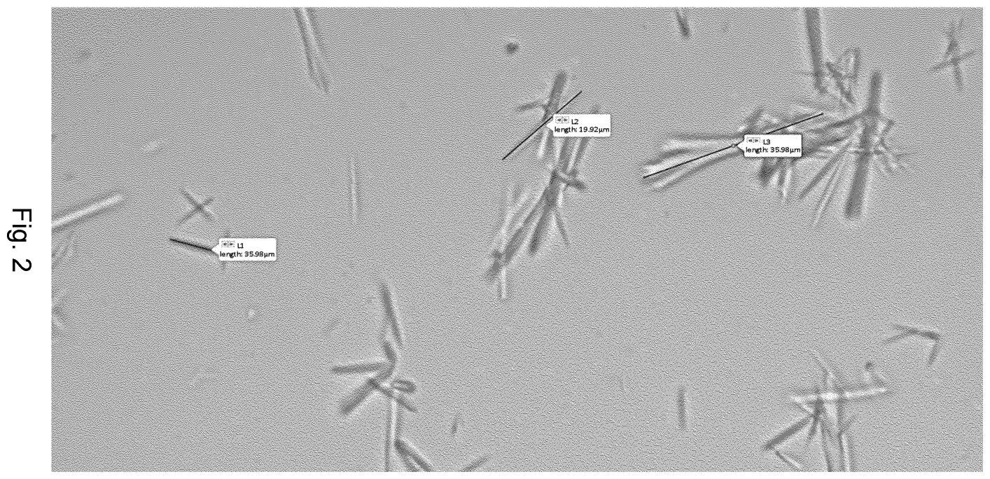

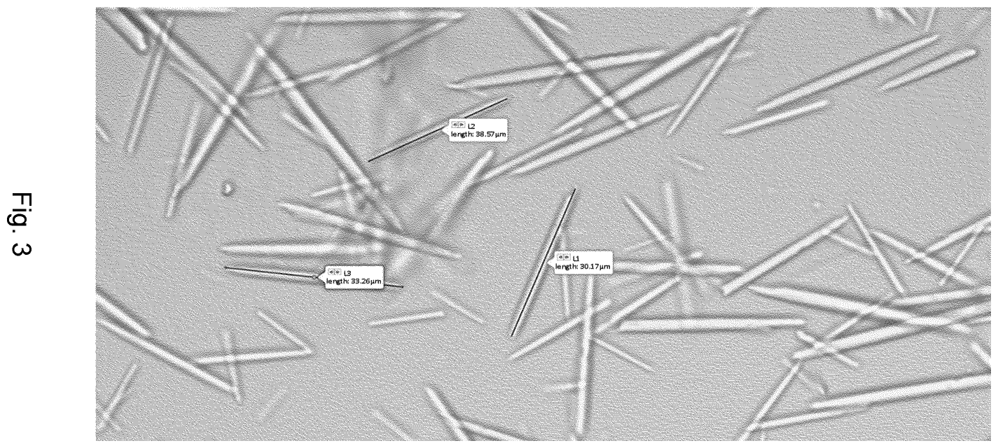

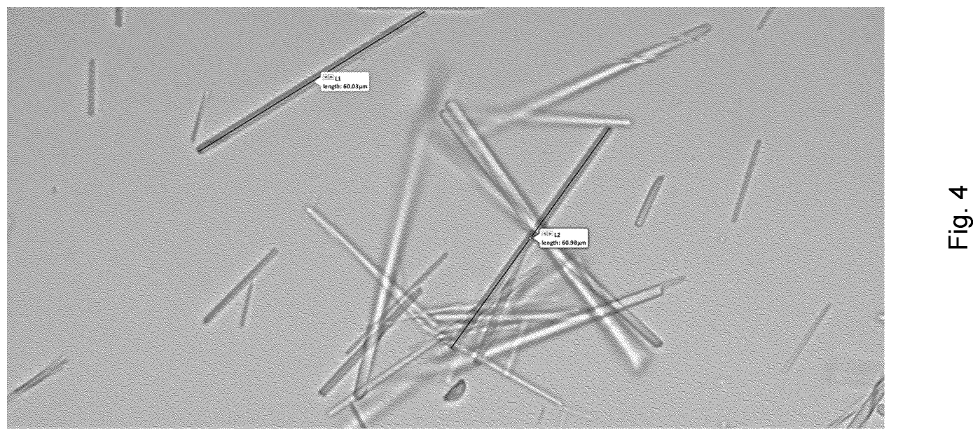

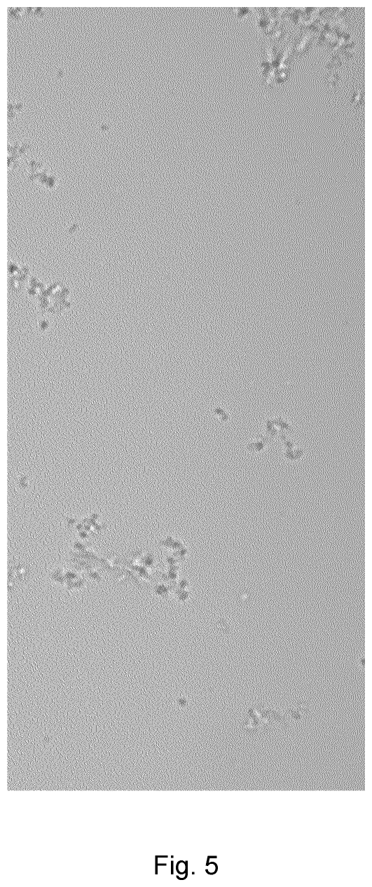

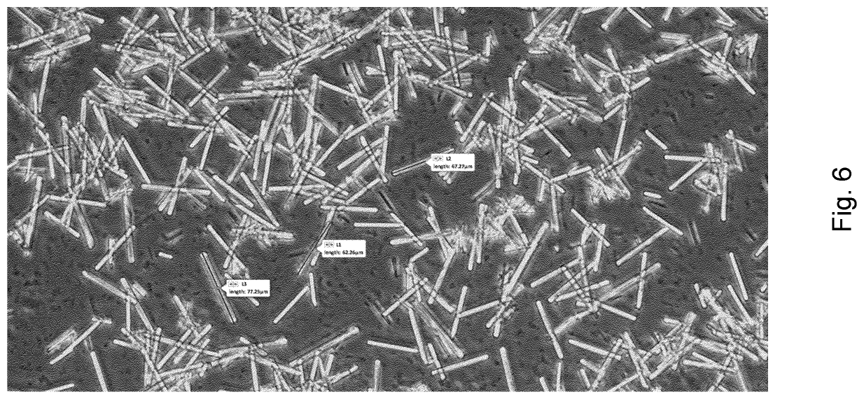

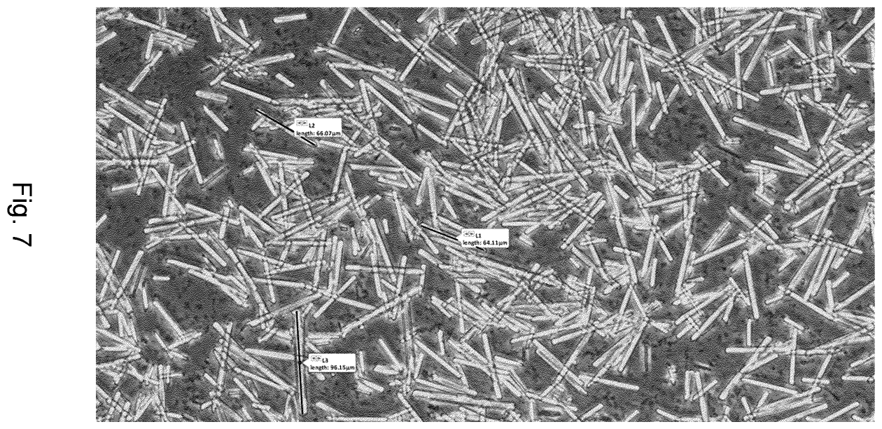

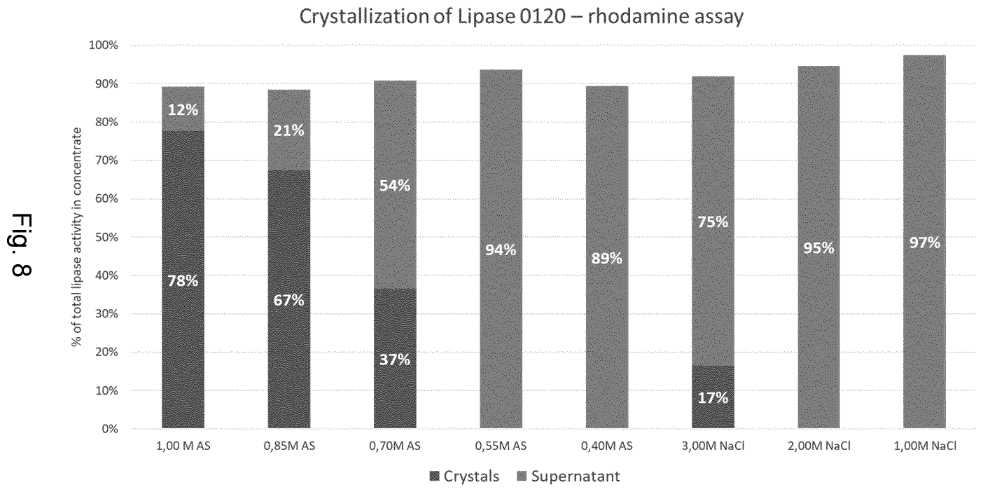

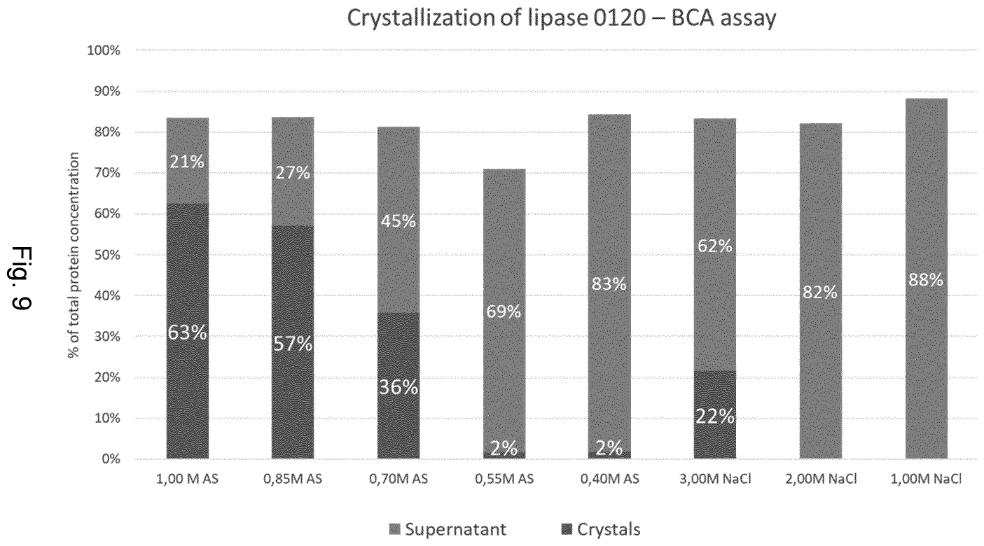

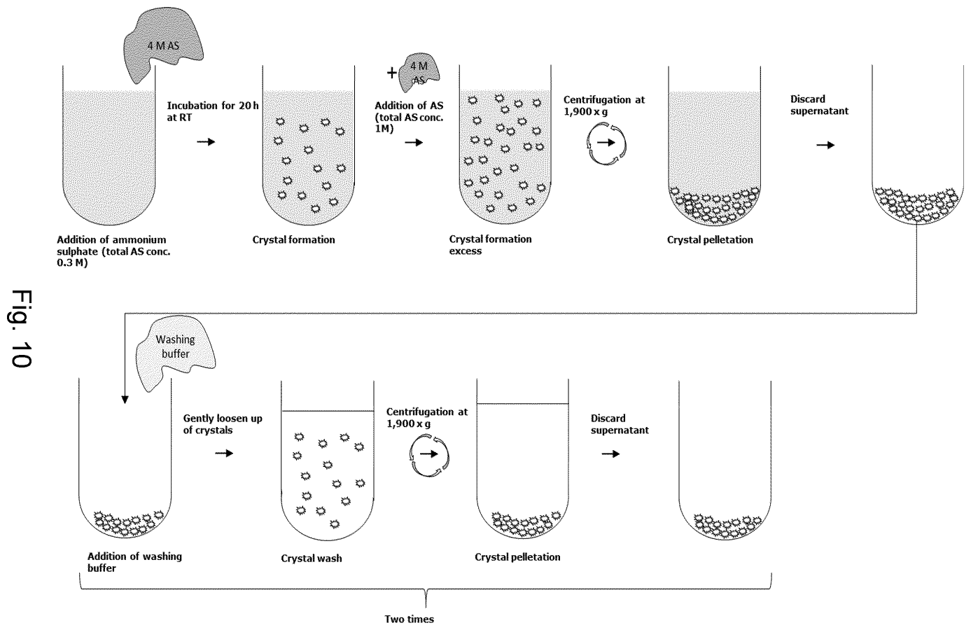

Further, without being bound to theory, one reasons for the fact that the method according to the invention delivers outstanding results without prior chromatographic purification of enzymes may lie in the fact that ciliates produce proteins which are characterized by a very simple and uniform glycosylation pattern. See FIG. 1 for an illustration.

Hence, in one embodiment, the protein is a glycosylated protein.

Generally, many proteins, including enzymes, require glycosylation to maintain activity and stability. Tetrahymena lipase 120 (discussed elsewhere herein) has for example two potential n-glycosylation sites (N61, motif is NV; N67, motif is NST).

In this regard, reference is made to, e.g., Chang et al (2007), who discuss that glycoproteins present special problems for structural genomic analysis because they often require glycosylation in order to fold correctly, whereas their chemical and conformational heterogeneity generally inhibits crystallization.

Interestingly, Chang et al propose the use of glycosylation processing inhibitors during protein expression, to avoid glycosylation and hence facilitate crystallization. Such process may yet seriously affect chemico-physical and physiological properties of enzymes thus treated, resulting in undesired loss of activity and/or stability.

Of course, enzymes can also be produced in prokaryotic expression systems. Because these systems do not glycosylate proteins at all, the proteins thus produced will likely encounter less problems upon crystallization, too. However, again, the complete lack of glycosylation may yet seriously affect chemico-physical and physiological properties of enzymes thus produced, resulting in undesired lack of activity and/or stability.

Under this aspect, and without being bound to theory, the fact that ciliates produce proteins which are characterized by a very simple and uniform glycosylation pattern (see FIG. 1) may indeed facilitate crystallization and make prior chromatographic purification steps or the use of glycosylation processing inhibitors obsolete.

According to one embodiment of the invention, the kosmotropic agent is at least one of
ammonium sulfate,
sodium dihydrogen phosphate, and/or
polyethylene glycol

According to one embodiment of the invention, the polyethylene glycol used has a size range of between ≥50 and ≤10000 g/mol.

In further embodiments, the polyethylene glycol used has a size range of ≥50 g/mol; ≥100 g/mol; ≥200 g/mol; ≥300 g/mol; ≥400 g/mol; ≥500 g/mol; ≥600 g/mol; ≥700 g/mol; ≥800 g/mol; ≥900 g/mol; ≥1000 g/mol; ≥1200 g/mol; ≥1400 g/mol; ≥1600 g/mol; ≥1800 g/mol; ≥2000 g/mol; ≥2200 g/mol; ≥2400 g/mol; ≥2600 g/mol; ≥2800 g/mol; ≥3000 g/mol; ≥3200 g/mol; ≥3400 g/mol; ≥3600 g/mol; ≥3800 g/mol; ≥4000 g/mol; ≥4200 g/mol; ≥4400 g/mol; ≥4600 g/mol; ≥4800 g/mol; ≥5000 g/mol; ≥5200 g/mol; ≥5400 g/mol; ≥5600 g/mol; ≥5800 g/mol; ≥6000 g/mol; ≥6200 g/mol; ≥6400 g/mol; ≥6600 g/mol; ≥6800 g/mol; ≥7000 g/mol; ≥7200 g/mol; ≥7400 g/mol; ≥7600 g/mol; ≥7800 g/mol; ≥8000 g/mol; ≥8200 g/mol; ≥8400 g/mol; ≥8600 g/mol; ≥8800 g/mol; ≥9000 g/mol; ≥9200 g/mol; ≥9400 g/mol; ≥9600 g/mol; ≥9800 g/mol and/or ≥10000 g/mol.

In further embodiments, the polyethylene glycol used has a size range of ≤10000 g/mol; ≤9800 g/mol; ≤9600 g/mol; ≤9400 g/mol; ≤9200 g/mol; ≤9000 g/mol; ≤8800 g/mol; ≤8600 g/mol; ≤8400 g/mol; ≤8200 g/mol; ≤8000 g/mol; ≤7800 g/mol; ≤7600 g/mol; ≤7400 g/mol; ≤7200 g/mol; ≤7000 g/mol; ≤6800 g/mol; ≤6600 g/mol; ≤6400 g/mol; ≤6200 g/mol; ≤6000 g/mol; ≤5800 g/mol; ≤5600 g/mol; ≤5400 g/mol; ≤5200 g/mol; ≤5000 g/mol; ≤4800 g/mol; ≤4600 g/mol; ≤4400 g/mol; ≤4200 g/mol; ≤4000 g/mol; ≤3800 g/mol; ≤3600 g/mol; ≤3400 g/mol; ≤3200 g/mol; ≤3000 g/mol; ≤2800 g/mol; ≤2600 g/mol; ≤2400 g/mol; ≤2200 g/mol; ≤2000 g/mol; ≤1800 g/mol; ≤1600 g/mol; ≤1400 g/mol; ≤1200 g/mol; ≤1000 g/mol; ≤900 g/mol; ≤800 g/mol; ≤700 g/mol; ≤600 g/mol; ≤500 g/mol; ≤400 g/mol; ≤300 g/mol; ≤200 g/mol; ≤100 g/mol and/or ≤50 g/mol.

According to one embodiment of the invention, the harvested cell culture fluid comprises between ≥0.05 and ≤200 g/l of protein.

In several embodiments, the harvested cell culture fluid comprises ≥0.05 g/l; ≥0.1 g/l; ≥0.15 g/l; ≥0.2 g/l; ≥0.3 g/l; ≥0.4 g/l; ≥0.5 g/l; ≥0.6 g/l; ≥0.7 g/l; ≥0.8 g/l; ≥0.9 g/l; ≥1 g/l; ≥2 g/l; ≥3 g/l; ≥4 g/l; ≥5 g/l; ≥6 g/l; ≥7 g/l; ≥8 g/l; ≥9 g/l; ≥10 g/l; ≥15 g/l; ≥20 g/l; ≥25 g/l; ≥30 g/l; ≥35 g/l; ≥40 g/l; ≥45 g/l; ≥50 g/l; ≥55 g/l; ≥60 g/l; ≥65 g/l; ≥ 70 g/l; ≥75 g/l; ≥80 g/l; ≥85 g/l; ≥90 g/l; ≥95 g/l; ≥100 g/l; ≥110 g/l; ≥120 g/l; ≥130 g/l; ≥140 g/l; ≥150 g/l; ≥160 g/l; ≥170 g/l; ≥180 g/l; ≥190 g/l; or ≥200 g/l of protein.

In several embodiments, the harvested cell culture fluid comprises ≤200 g/l; ≤190 g/l; ≤180 g/l; ≤170 g/l; ≤160 g/l; ≤150 g/l; ≤140 g/l; ≤130 g/l; ≤120 g/l; ≤110 g/l; ≤100 g/l; ≤95 g/l; ≤90 g/l; ≤85 g/l; ≤80 g/l; ≤75 g/l; ≤70 g/l; ≤65 g/l; ≤60 g/l; ≤55 g/l; ≤50 g/l; ≤45 g/l; ≤40 g/l; ≤35 g/l; ≤30 g/l; ≤25 g/l; ≤20 g/l; ≤15 g/l; ≤10 g/l; ≤9 g/l; ≤8 g/l; ≤7 g/l; ≤6 g/l; ≤5 g/l; ≤4 g/l; ≤3 g/l; ≤2 g/l; ≤1 g/l; ≤0.9 g/l; ≤0.8 g/l; ≤0.7 g/l; ≤0.6 g/l; ≤0.5 g/l; ≤0.4 g/l; ≤0.3 g/l; ≤0.2 g/l; ≤0.15 g/l; ≤0, 1 g/l; or ≤0.05 g/l of protein.

According to several embodiments of the invention, for efflorescing the protein,
a pH of between ≥5 and ≤8 is established in the medium, and/or
a temperature of between ≥10 and ≤40° C. is established.

According to one embodiment of the invention, for the incubation of the harvested cell culture fluid, a kosmotropic salt concentration is established in the range of between ≥50 and ≤2500 mM.

In several embodiments, the kosmotropic salt concentration is established to be ≥50 mM; ≥ 100 mM; ≥150 mM; ≥200 mM; ≥250 mM; ≥300 mM; ≥350 mM; ≥400 mM; ≥450 mM; ≥500 mM; ≥550 mM; ≥600 mM; ≥650 mM; and ≥700 mM.

In several embodiments, the kosmotropic salt concentration is established to be ≤2500 mM; ≤2300 mM; ≤2100 mM; ≤2000 mM; ≤1900 mM; ≤1800 mM; ≤1700 mM; ≤1600 mM; ≤1500 mM; ≤1400 mM; ≤1300 mM; ≤1200 mM; ≤1100 mM; and ≤1000 mM.

In several embodiments, the kosmotropic salt concentration is established in the range of between ≥50 mM and ≤2500 mM; between ≥100 mM and ≤2300 mM; between ≥150 mM and ≤2100 mM; between ≥200 mM and ≤2000 mM; between ≥250 mM and ≤1900 mM; between ≥300 mM and ≤1800 mM; between ≥350 mM and ≤1700 mM; between ≥400 mM and ≤1600 mM; between ≥450 mM and ≤1500 mM; between ≥500 mM and ≤1400 mM; between ≥550 mM and ≤1300 mM; between ≥600 mM and ≤1200 mM; between ≥ 650 mM and ≤1100 mM; between ≥700 mM and ≤1000 mM;

Preferably, these concentration ranges apply to ammonium sulfate and/or to sodium dihydrogen phosphate.

In one embodiment, for the crystallization of an amylase, a kosmotropic salt concentration of ≥100 mM is used. In one embodiment, for the crystallization of a lipase, a kosmotropic salt concentration of ≥300 mM is used. Preferably, these concentration ranges apply to ammonium sulfate and/or to sodium dihydrogen phosphate.

In several embodiments, kosmotropic salt concentration is established in the range of between ≥700 mM and ≤1 M. Preferably, these concentration ranges apply to ammonium sulfate and/or to sodium dihydrogen phosphate.

According to one embodiment of the invention, for the incubation of the harvested cell culture fluid, a polyalcohol concentration is established in the range of between ≥2 and ≤25% w/v)

In several embodiments, a polyalcohol concentration is established which is ≥2% w/v; ≥2.5%; ≥3%; ≥3.5%; ≥4%; ≥4.5%; ≥5%; ≥5.5%; ≥6%; ≥6.5%; ≥7%; ≥7.5%; ≥8%; ≥ 8.5%; ≥9%; ≥9.5%; ≥10%; ≥10.5%; ≥11%; ≥11.5%; ≥12%; ≥12.5%; ≥13%; ≥13.5%; ≥14%; ≥14.5%; ≥15%; ≥15.5%; ≥16%; ≥16.5%; ≥17%; ≥17.5%; ≥18%; ≥18.5%; ≥19%; ≥19.5% and/or ≥20% w/v.

In several embodiments, a polyalcohol concentration is established which is ≤25% w/v; ≤24.5%; ≤24%; ≤23.5%; ≤23%; ≤22.5%; ≤22%; ≤21.5%; ≤21%; ≤20.5%; ≤20%; ≤19.5%; ≤19%; ≤18.5%; ≤18%; ≤17.5%; ≤17%; ≤16.5%; ≤16%; ≤15.5%; ≤15%; ≤14.5%; ≤14%; ≤13.5%; ≤13%; ≤12.5%; ≤12%; ≤11.5%; ≤11%; ≤10.5% and/or ≤10% w/v.

Preferably, these concentration ranges apply to polyethylene glycol.

In several embodiments, the polyalcohol is PEG 4000 (4000 g/mol). Preferably, a concentration of 6% w/v is established.

In several embodiments, the polyalcohol is PEG 6000 (6000 g/mol). Preferably, a concentration of 6% w/v is established.

The above concentration ranges are in no way binding, because the inventors have realized that, the higher the protein concentration in the medium, the lower the required concentration of the kosmotropic agent is.

According to one embodiment of the invention, the incubation of the harvested cell culture fluid with the kosmotropic agent is carried out over a period of between ≥10 min and ≤36 hrs.

According to one embodiment of the invention, a pre-harvest cell culture fluid is subjected to one or more filtration and/or centrifugation steps prior to step a), to obtain the harvested cell culture fluid.

Such filtration may include at least one of the following:
Microfiltration, e.g., in a hollow fiber cross flow filtration system, to remove cells from the CCF (size exclusion in the range of 0.4-0.8 μm)
Ultrafiltration with optional subsequent diafiltration, to remove smaller peptides and impurities.

Such centrifugation may include the use of a centrifuge or a centrifugal separator, like e.g. provided by Westfalia Separator. In one embodiment, at least one filter that is applied has a molecular weight cut off (MWCO) that is adapted to ten respective protein to be purified.

In one embodiment, at least one filter that is applied has a molecular weight cut off (MWCO) of ≤50 kd.

In further embodiments, at least one filter that is applied has a molecular weight cut off (MWCO) of ≤49 kDa; ≤48 kDa; ≤47 kDa; ≤46 kDa; ≤45 kDa; ≤44 kDa; ≤43 kDa; ≤42 kDa; ≤41 kDa; ≤40 kDa; ≤39 kDa; ≤38 kDa; ≤37 kDa; ≤36 kDa; ≤35 kDa; ≤34 kDa; ≤33 kDa; ≤32 kDa; ≤31 kDa; ≤30 kDa; ≤29 kDa; ≤28 kDa; ≤27 kDa; ≤26 kDa; ≤25 kDa; ≤24 kDa; ≤23 kDa; ≤22 kDa; ≤21 kDa; ≤20 kDa; ≤19 kDa; ≤18 kDa; ≤17 kDa; ≤16 kDa; ≤15 kDa; ≤14 kDa; ≤13 kDa; ≤12 kDa; ≤11 kDa; ≤10 kDa; ≤9 kDa and preferably ≤8 kDa.

In this context, it is important to understand that the molecular weight cut off is adapted to the molecular weight of the protein without signal peptide. Further, it has been shown in the past that enzymes can form enzymatically active fragments which retain enzyme activity, like e.g. Protease CCP5 (which forms an active fragment that has 12 kDa).

According to one embodiment of the invention, the ciliate host is Tetrahymena sp.

In one embodiment, the ciliate host is Tetrahymena thermophila. Note, in this context, that Tetrahymena thermophila, has been used as expression hosts for the production of heterologous or homologous protein expression, as e.g. described in Weide et al. 2006, as well as in EP1350838 (U.S. Pat. No. 6,962,800), the content of which is incorporated herein by reference for enablement purposes.

Generally, methods for the transformation of ciliates including Tetrahymena, which can be used in the context of the present invention, comprise, among others, microinjection, electroporation and particle bombardment, and are, for example, described in Tondravi & Yao (1986), Gaertig & Gorovsky (1992) and Cassidy-Hanley et al (1997).

Methods for transformation and heterologous protein expression have been described for a few protists (WO 00/58483 and WO 00/46381). The generation of mitotically stable transformants of the ciliate Tetrahymena thermophila can be achieved after transfection either of the somatic macronucleus or the generative micronucleus by microinjection, electroporation or by particle bombardment.

Selection of the transformants can be performed using different selection markers like the neomycin resistance (Weide et al. 2006, BMC) and the integration of the heterologous genes by homologous DNA recombination, which results in stable thymidin-auxotrophic Tetrahymena cells (Weide et al. 2006, BMC). In addition, the use of blasticidin S (Weide et al. 2007, BMC) or paclitaxcel (WO 00/46381) resistance has also been considered.

Promoters suitable for protein expression in ciliates are, for example, disclosed in US2008261290A1 which is also registered for the applicant of the present invention, the content of which shall be incorporated herewith by reference. Therein, a heat-inducible promoter and a metallothionein-promoter are disclosed which can also be used for the purposes of the present invention.

According to one embodiment of the invention, the homologous or heterologous expression is under control of
a metallothionein gene (MTT1) inducible promoter, and/or
a heat inducible promoter.

Said MTT1 inducible promoter is for example disclosed in WO2003006480A1 (US20030027192A1), the content of which is incorporated herein for enablement purposes.

Said heat inducible promoter is for example disclosed in EP1902138B1 (U.S. Pat. No. 7,723,506B2), the content of which is incorporated herein for enablement purposes

According to one embodiment of the invention, the protein is an enzyme

According to further embodiments the enzyme is at least one selected from the group consisting of
lipase
protease
amylase
glutenase
glutamine-specific cysteine protease
prolyl endopeptidases
saccharase, and/or
lactase.

A lipase, amylase or protease can for example be used in pancreatic enzyme replacement therapy (PERT), for the treatment of pancreatic exocrine insufficiency. Pancreatic exocrine insufficiency can have the following causes:

| Cause | Mechanism |
|---|---|
| Chronic pancreatitis | Permanent structural damage of ducts and parenchyma |
| Cystic fibrosis | Dysfunctional pancreatic secretion secondary to CFTR |
| | mutations |
| Main pancreatic duct obstruction | Decreased secretion of pancreatic enzymes |
| Pancreatic resection | Decreased secretion of pancreatic enzymes |
| Gastric resection | Decreased hormonal stimulation, rapid transit, |
| | inadequate mixing of chyme with pancreatic enzymes |
| Short bowel syndrome | Decreased hormonal stimulation, rapid transit, |
| | inadequate mixing of chyme with pancreatic enzymes |
| Hereditary hemochromatosis | Iron deposition in the pancreas |
| Celiac disease | Small bowel mucosal disease leads to decreased CCK |
| | mediated pancreatic stimulation |
| Zollinger-Ellison syndrome | Gastrinoma causes inactivation of pancreatic enzymes |
| | via increased gastric acid |

In such therapy, patients oftentimes need large amounts of enzyme, which is administered orally. The standard of care is a product made from pork pancreas homogenate (i.e., a highly complex product with varying lipase content), granulated and provided as capsules, of which patients have to take in average 2.5 g (8 capsules with 300 mg each) per day or even more (it has been reported that some patients have to take up to 50 capsules). This results in about 1 kg or product per year, which emphasizes the fact that purification by chromatography would excessively drive product costs.

According to one embodiment of the invention, the enzyme is
a) Tetrahymena lipase according to any one of SEQ ID NOs 1-3 or 8-10,
b) Tetrahymena amylase according to SEQ ID NOs 4 or 11,
c) Tetrahymena protease according to any of SEQ ID NOs 5-7, 12-14 or 15-17, or
d) a variant of any one of the above enzymes having
   (i) at least 90% amino acid sequence identity therewith, wherein said variant retains enzyme functionality, and/or
   (ii) between 1 and 3 amino acid residues removed or added at the N-terminus and/or the C terminus

SEQ ID NO 1 is the sequence of Tetrahymena lipase 120 as published under the UniProt Identifier: Q237S4 (TTHERM_00320120). It has already been discussed in, inter alia, WO2016116600A1 (U.S. Ser. No. 10/590,402B2), and is currently in preclinical evaluation for pancreatic enzyme replacement therapy (PERT).

The further enzymes are detailed below. Note that the respective SEQ ID NOs 1-7 refer to the enzymes with the signal peptides. SEQ ID NOs 8-14 refer to the enzymes without the signal peptides. SEQ ID NOs 15-17 refer to the protease enzymes without the propeptide.

| | | | | | Molecular |
|---|---|---|---|---|---|
| | | | | Molecular | weight (w/o |
| | | UniProt identifier/ | Molecular | weight (w/o | signal peptide |
| SEQ ID | Enzyme | Gene Model identifier | weight | signal peptide) | and propetide) |
| 1, 8 | Lipase 120 | Q237S4/ | 32.46 kDa | 30.712 kDa | |
| | | TTHERM_00320120 | | | |
| 2, 9 | Lipase 120 | Q237S3/ | 32.974 kDa | 31.011 kDa | |
| | | TTHERM_00320130 | | | |
| 3, 10 | Lipase 230 | Q237R4/ | 35.199 kDa | 33.195 kDa | |
| | | TTHERM_00320230 | | | |
| 4, 11 | Amylase 610 | I7LW22/ | 55.363 kDa | 53.394 kDa | |
| | | TTHERM_00411610 | | | |
| 5, 12, | Protease CCP1 | I7MB12/ | 35.902 kDa | 33.976 kDa | 22.41 kDa |
| 15 | | TTHERM_00683060 | | | |
| 6, 13, | Protease CCP2 | I7MB11/ | 34.968 kDa | 32.759 kDa | 22.322 kDa |
| 16 | | TTHERM_00683010 | | | |
| 7, 14, | Protease CCP5 | I7MCX6/ | 35.43 kDa | 32.855 kDa | 22.222 kDa |
| 17 | | TTHERM_ 00530660 | | | |

“Percentage of sequence identity” as used herein, is determined by comparing two optimally aligned biosequences (amino acid sequences or polynucleotide sequences) over a comparison window, wherein the portion of the corresponding sequence in the comparison window may comprise additions or deletions (i.e., gaps) as compared to the reference sequence, which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides that are substantially identical to the polypeptides exemplified herein. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

According to one embodiment, the method further comprises the step of solubilizing the effloresced protein. In such way, the thus purified protein is again transferred into solution, This may be helpful for applications where soluble protein is required, e.g., for parenteral administration. Other applications, like e.g. enteral administration, may use the effloresced form of the protein.

According to another aspect of the invention, a pharmaceutical intermediate is provided which has been obtained with a method according to the above description. According to a general definition, pharmaceutical intermediates are raw drugs or prodrugs obtained by synthesis or biofermentation which are used as raw materials for the production of pharmaceutic preparations which then can be administered to patients. For that purpose, a pharmaceutical intermediate must undergo further molecular changes or processing.

In the context of the present invention, such intermediate is therefore defined as a product which has been obtained, directly or indirectly, by the method according to the above description, but is per se not yet ready for administration to a patient. This means that said intermediate is then used to produce a pharmaceutic preparation. It can be considered a pro-drug in some aspects. Such intermediate can adopt the solubilized form or the effloresced (=crystallized) from as described above.

Therefore. according to one embodiment, the pharmaceutical intermediate comprises a solubilized form of the effloresced protein or the effloresced protein per se.

According to another aspect of the invention, a purified protein or pharmaceutical intermediate is provided that has been obtained with a method according to the above description.

According to another aspect of the invention, a purified, crystallized protein or pharmaceutical intermediate is provided which comprises a ciliate-type glycosylation pattern. As discussed herein, ciliates produce proteins which are characterized by a very simple and uniform glycosylation pattern, that is unique relative to other taxa.

In particular, the ciliate-type glycosylation pattern is at least one of
devoid of fucose
devoid of xylose
poor in mannose
devoid of galactose
devoid of N-acetylneuraminic acid, and/or
devoid of N-glyclylneuraminic acid,

See FIG. 1 for an illustration. Such protein can for example be obtained with a method according to the above description.

According to one embodiment of the invention, the intermediate or protein is or comprises at least one of
a) Tetrahymena lipase according to any one of SEQ ID NOs 1-3 or 8-10,
b) Tetrahymena amylase according to SEQ ID NOs 4 or 11,
c) Tetrahymena protease according to any of SEQ ID NOs 5-7, 12-14 or 15-17, or
d) a variant of any one of the above enzymes having
   (i) at least 90% amino acid sequence identity therewith, wherein said variant retains enzyme functionality, and/or
   (ii) between 1 and 3 amino acid residues removed or added at the N-terminus and/or the C terminus

According to another embodiment of the invention, the intermediate or protein is or comprises an enzyme selected from the group shown in the following table:

| Enzyme | Disease |
|---|---|
| glutenase, glutamine-specific | celiac sprue |
| cysteine protease or | |
| prolyl endopeptidases | |
| saccharase (e.g., sucrosidase = | sucrose intolerance or genetic sucrase- |
| saccharase from yeast) | isomaltase deficiency (GSID) |
| lactase (e.g., β-galactosidase) | lactose intolerance |

These enzymes are used for oral enzyme replacement therapy, and just like in pancreatic enzyme replacement therapy, they are needed in large quantities, making cost effective expression and purification techniques advantageous—like the crystallization process described herein.

According to one embodiment of the invention, the intermediate or protein is provided in crystalline form.

According to one embodiment of the invention, the intermediate or protein has a purity grade of ≥80%.

According to one embodiment of the invention, the protein crystals of the intermediate or protein have a length of between ≥2 and ≤1000 μm.

According to one embodiment of the invention, the protein crystals of the intermediate or protein have a diameter of between ≥0.05 and ≤10 μm.

According to another aspect of the invention, a pharmaceutic preparation comprising the intermediate or protein according to the above description is provided, and optionally further one or more further pharmaceutically acceptable excipients.

According to several embodiments of the invention, the protein is an enzyme.

According to further embodiments of the invention, the enzyme is at least one selected from the group consisting of
lipase
protease
amylase
glutenase
glutamine-specific cysteine protease
prolyl endopeptidases
saccharase, and/or
lactase.

In further embodiments, the enzyme is
a) Tetrahymena lipase according to any one of SEQ ID NOs 1-3 or 8-10,
b) Tetrahymena amylase according to SEQ ID NOs 4 or 11,
c) Tetrahymena protease according to any of SEQ ID NOs 5-7, 12-14 or 15-17, or
d) a variant of any one of the above enzymes having
   (i) at least 90% amino acid sequence identity therewith, wherein said variant retains enzyme functionality, and/or
   (ii) between 1 and 3 amino acid residues removed or added at the N-terminus and/or the C terminus

The possibility of a modular assembly of lipase, protease and amylase activity allows the adaptation of the preparation to patients with different conditions and thus different needs of medications. For example, high amylase content is undesirable for children with mucoviscidose. Proteases are contraindicated in patients with acute pancreatitis or active episodes of chronic pancreatitis. And fourthly, the preparation, in contrast to lipases from fungi is activated by bile acids in physiologic concentrations.

According to another aspect of the invention, the enzyme or the pharmaceutic preparation according to the above description is provided (for the manufacture of a medicament) for use in the treatment of a human or animal subject
being diagnosed for,
suffering from or
being at risk of developing
   a disorder caused by enzyme deficiency or dysfunction.

This language is deemed to encompass both the swiss type claim language accepted in some countries (in this case, brackets are deemed absent) and EPC2000 language (in this case, brackets and content within the brackets is deemed absent).

According to another aspect of the invention, a method for treating or preventing a disorder caused by enzyme deficiency or dysfunction is provided, which method comprises administration, to a human or animal subject, the enzyme or the pharmaceutic preparation according to the above description in a therapeutically sufficient dose.

According to further embodiments, the disorder caused by enzyme deficiency or dysfunction is at least one selected from the group consisting of
a lipid digestion deficiency and/or a digestive disorder
disorder caused by enzyme deficiency or dysfunction.
pancreatic exocrine insufficiency can have the following causes:
chronic pancreatitis
cystic fibrosis
main pancreatic duct obstruction
pancreatic resection
gastric resection
short bowel syndrome
hereditary hemochromatosis
celiac disease
Zollinger-Ellison syndrome

In one embodiment, in said use the digestive disorder is exocrine pancreatic insufficiency (EPI). Such exocrine pancreatic insufficiency can be caused, inter alia, by cystic fibrosis, blockage of the pancreatic duct, or pancreatectomy.

According to one other embodiment, the inflammatory condition is chronic inflammation of the pancreas (pancreatitis) or inflammatory bowel disease

Other digestive disorders that can be treated encompass steatorrhea, celiac disease or indigestion

EPI is the inability to properly digest food due to a lack of digestive enzymes made by the pancreas. EPI is found in humans afflicted with cystic fibrosis and Shwachman-Diamond Syndrome, and is caused by a progressive loss of the pancreatic cells that make digestive enzymes; loss of digestive enzymes leads to maldigestion and malabsorption of nutrients from normal digestive processes. Chronic pancreatitis is the most common cause of EPI in humans.

Steatorrhea is the presence of excess fat in feces. Stools may also float due to excess lipid, have an oily appearance and can be especially foul-smelling. An oily anal leakage or some level of fecal incontinence may occur. There is increased fat excretion, which can be measured by determining the fecal fat level. The definition of how much fecal fat constitutes steatorrhea has not been standardized.

Indigestion is a condition in which patients suffer bloating, gas, and fullness following a high fat meal. These symptoms are commonly associated with irritable bowel syndrome (IBS), so some researchers speculate that pancreatic enzymes might help treat symptoms of IBS. No studies have been done, however.

In one embodiment, in said use the lipid digestion deficiency is Lipoprotein lipase deficiency, which is a condition caused by mutation in the gene which codes lipoprotein lipase.

According to still another embodiment of the present invention, the use of the combination of two or more lipase enzymes according the above description, or of a pharmaceutical preparation comprising the latter, for the treatment of Cystic fibrosis is provided.

Cystic fibrosis is an inherited condition that causes the body to produce abnormally thick, sticky mucus. Patients often have nutritional deficiencies because mucus blocks pancreatic enzymes from getting to the intestines. Taking lipases helps improve the nutrition these patients get from food.

According to further embodiments, the disorder caused by enzyme deficiency or dysfunction is at least one selected from the group consisting of
celiac sprue
sucrose intolerance or genetic sucrase-isomaltase deficiency (GSID), and/or
lactose intolerance

As discussed elsewhere herein, these diseases can be advantageously treated by oral enzyme replacement therapy with the respective enzymes as produced with the method disclosed herein.

According to another aspect of the invention, a dosage unit comprising the protein or the pharmaceutic preparation according to the above description is provided.

In one embodiment, such dosage unit is provided in an entity that is suitable for oral administration.

In several embodiments, said dosage unit is a capsule, tablet, or a sachet.

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′→3′.

1. Crystallization of Lipase from T. thermophila Using Ammonium Sulphate

### Materials and Methods

### Media:

Standard complex medium
   Paromomycin concentration for Fermentation:
2 μg/ml Paromomycin

### Buffers:

ammonium sulphate stock solution
20 mM phosphate-citrate
4 M ammonium sulphate
pH 6.0

### Washing Buffer

20 mM phosphate-citrate
0.7 M ammonium sulphate
pH 7.0

### Citrate/Phosphate Buffer

20 mM phosphate-citrate
pH 7.0

### Machines:

| Equipment | Designation | Supplier |
|---|---|---|
| Sorvall Lynx 6000 Centrifuge | Crystal separation | Thermo Scientific |
| Motic microscope AE2000 | visual evaluation | Motic Deutschland |
| | | GmbH |
| Synergy H1 | Fluorescence detection | BioTek Instruments Inc. |
| Techfors T 900 | Fermentation/Cell Cultivation | Infors, Switzerland |
| Spectrum PES; Hollow Fiber | Cell free harvest | Spectrum Laboratories |
| Modul | | Inc., Rancho Dominguez, |
| | | California, USA |
| Sartocon Slice Hydrosart | Filtrate Concentration | Sartorius, Göttingen, |
| | | Germany |
| Biolistic gun | PDS-1000/He Biolistic ® | BioRad Laboratories |
| | Particle Delivery System | GMBH Hercules- |
| | | California, USA |

### Software

| Equiment | Designation | Supplier |
|---|---|---|
| Motic Images | Visual evaluation | Motic Deutschland |
| Plus 3.0 | | GmbH |
| Protparam | Protein sequence evaluation | Gasteiger et al. |

### Transformation

For the generation of the cell substrate (Tt_pAX_hn_MTT1_Ttherm_00320120_K12) the episomal vector pAX_hn_MTT1_Ttherm_00320120 (provided by Cilian AG) was inserted in conjugating parental cells T. thermophila B 1868.7 and T. thermophila SB 1969 by the method of biolistic transformation which was performed as previously described in Cassidy-Hanley et al. (1997).

### Cell Culture/Fermentation

Batch fermentation of T. thermophila was carried out in a Techfors T900 fermenter (Infors, Bottmingen-Basel, Switzerland) at a 600-1 scale. The fermenter was inoculated with 10×10³ cells/ml and cells were grown in a modified SPP medium. Temperature was maintained at 30° C. and pO₂ was controlled at 20% of the air saturation level by stirrer speed (50-105 rpm) and air flow (0.1-0.3 vvm). The pH value was regulated to pH 7.0 during the fermentation process. Lipase expression was induced by adding 10 μg/ml cadmium chloride to the culture in the log phase.

### Harvest and Filtration/USP

After 24 h postinduction, the culture broth was harvested cell free by filtration with a hollow fiber module (Spectrum PES, 0.5 mm lumen, 2.9 m² area, 0.5 μm cut off; Spectrum Laboratories Inc., Rancho Dominguez, California, USA), producing 550 l of supernatant from 600 l culture. The filtrate was concentrated to 6 l using Sartocon Slice Hydrosart tangential flow cassettes (30 kDa cut off; Sartorius, Göttingen, Germany). The concentrate was washed with buffer (Citrate-phosphate, pH7.0) at the end of the concentration and frozen immediately at −80° C.

### Crystallization Experiments

The lipase protein crystallization was carried out by adding an appropriate crystallization reagent to the protein concentrate. The concentrated and diafiltrated fermentation broth possessed a protein concentration of 46.69 mg/ml. The concentrate was mixed with the ammonium sulphate stock solution to a final ammonium sulphate concentration of 0.3 M (FIG. 1). This suspension was incubated for 20 hours at room temperature. The ammonium sulphate concentration of the suspension was then increased to 1 M. The prepared suspension was further incubated for 2 hours at room temperature. The crystallization broth was then centrifuged (Sorvall Lynx 6000 Centrifuge; Thermo Scientific™) at 1,900×g for 10 min. The supernatant was discarded, and the remaining crystal pellet was laminated and loosen in washing buffer. This suspension was subsequently centrifuged again for 10 min at 1,900×g. All in all, the crystal pellet was washed two times and the supernatants have been discarded. The final centrifugation step was carried out at 2,500×g for 20 min. The wet crystal pellet was then stored at −20° C. for further investigations.

### Photomicrography

The photomicrography was carried out by using the Motic microscope AE2000 and the corresponding evaluation software Motic Images Plus 3.0. 20 μl of the crystal suspension was transferred onto a microscopy slide and covered with a glass slide. The prepared suspension was visually inspected using several amplifications (10×, 20× and 40×). The length and width were measured using the evaluation software.

### Rhodamine Assay

The lipolytic activity was measured based on the Rhodamine-Triglyceride-Agarose assay published by Jette et al. In contrast to the original protocol the Triglyceride-Agarose mixture has been replaced by olive oil as substrate. The active lipase cleaves triglycerides into glycerol and free fatty acids. These fatty acids bind to rhodamine B and can be detected with an excitation wavelength of 485 nm and an emission wavelength of 535 nm. Samples must be diluted facilitating an acceptable extinction. Thus, these diluted samples are mixed with the olive oil rhodamine mixture in a micro titer plate scale. The samples were then incubated for 20 min in the micro titer plate reader (Synergy H1; BioTek Instruments, Inc.) at 30° C. The fluorescence was measured for 20 min every 50 sec. Finally, the measured activity was calculated according to the activity of a reference standard.

### Lambert-Beer Assay

Presuming a high lipase protein purity, the protein concentration was determined based on the Lambert-Beer law. The absorbance was measured at 280 nm and the based on the equation (i) the protein concentration has been calculated. The extinction co-efficient was determined by using the in silico prediction tool Protparam.

E_{λ}=ε_{λ}*c*d (i)
Equation 1: ε_{λ}=molar absorptivity; c=molar concentration; d=layer thickness
   2. Crystallization of Alpha-Amylase from T. thermophila Using PEG 4000

a-Amylase (TTHERM_00411610) from T. thermophila was crystallized from an amylase rich protein concentrate (95 g/L) buffered with 20 mM phosphate-citrate (pH 7) obtained by fermentation of recombinant T. thermophila using PEG 4000 and the subsequent DSP.

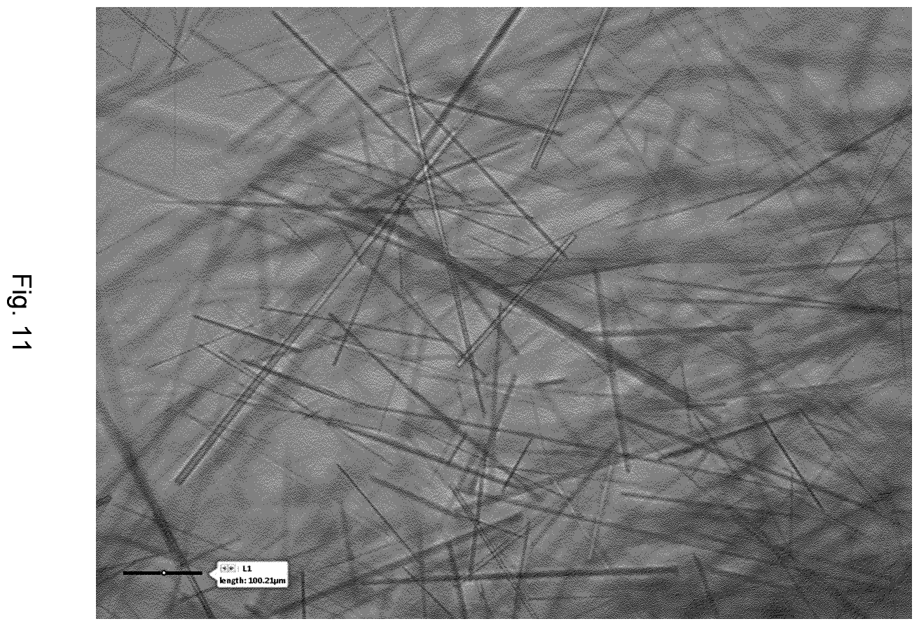

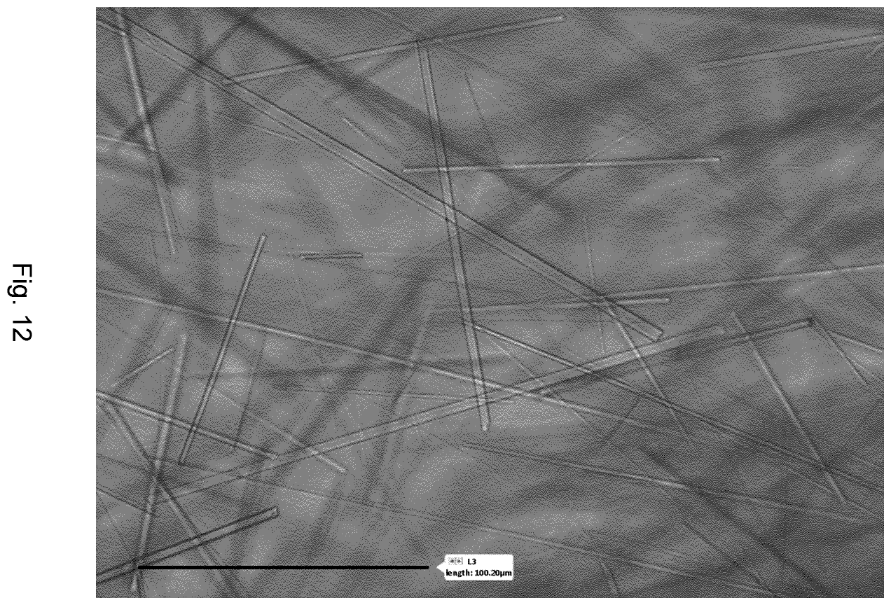

The crystallization was started by slowly adding 25% (v/v) of a PEG 4000 stock solution (40% (w/v) PEG 4000 in 20 mM phosphate-citrate buffer, pH 7) into the gently stirred amylase concentrate resulting in a final PEG 4000 concentration of 6% (w/v). After the solution was properly mixed, the mixture was incubated at room temperature (20-22° C.) for 93 h without agitation. Samples were withdrawn daily and microscopically examined for the formation of crystals. While no crystallization could be observed within the first three days, after 93 h large needle-shaped crystals with a length of up to about 650 μm and width of up to about 5 μm were visible (FIGS. 11 and 12).

The crystals were pelleted by centrifugation (10 min, 1500 rcf, RT) and resolubilized in H₂O. The crystal solution as well as the centrifugation supernatant were subjected to a determination of the amylase activity using the Phadebas Amylase Test (Phadebas AB, Kristianstad, Sweden) according to the recommendations of the manufacturer. About 67% of the total amylase activity were found in the resolved crystal pellet, while about 33% remained in the supernatant.

3. Crystallization of Lipase from T. thermophila Using Sodium Dihydrogen Phosphate

### Buffers:

NaH₂PO₄ Stock Solution
20 mM K₂HPO₄
2.5 M NaH₂PO₄
pH 7.0

### Dissolving Buffer

10 mM K₂HPO₄
pH 7.0

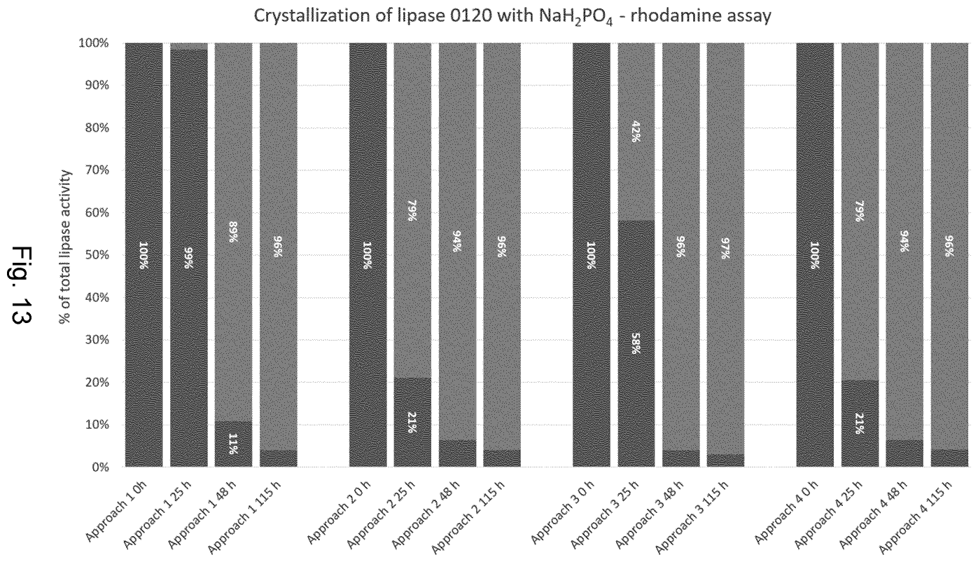

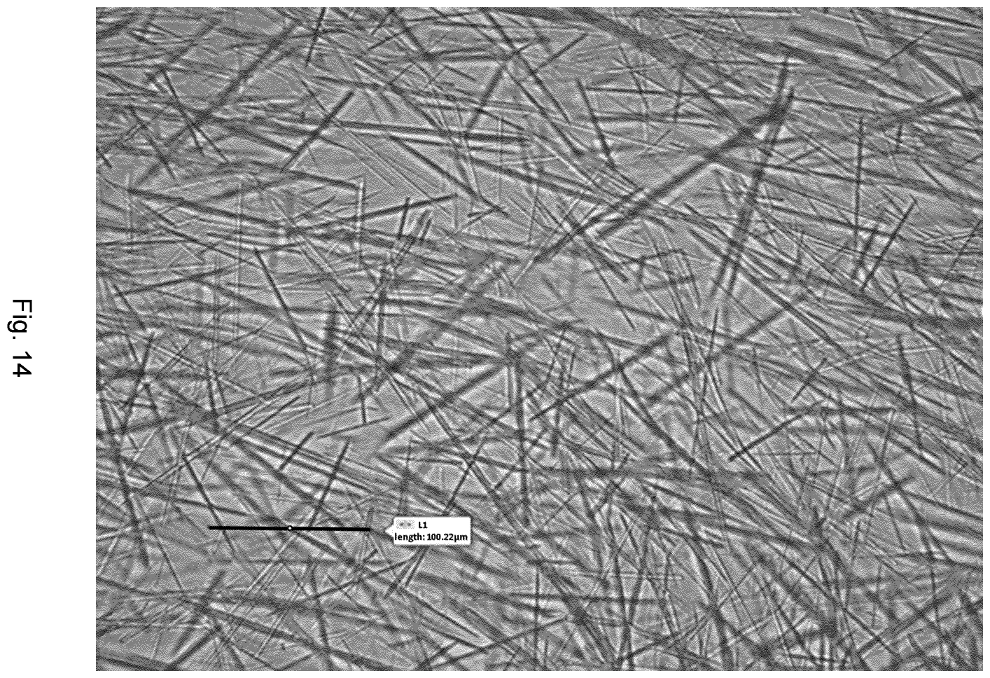

Harvested cell culture fluid (HCCF) with overproduced Tetrahymena lipase 120 was filtered to remove any undissolved components and then concentrated to 55 g/L (approach 1), 40 g/L (approach 2), 25 g/L (approach 3) and 16 g/L (approach 4). The four samples were adjusted to reach a final concentration of 0.3 M NaH₂PO₄ (approach 1), 0.5 M NaH₂PO₄ (approach 2 and 3) and 0.7 M NaH₂PO₄ (approach 4). After 25 h the NaH₂PO₄ concentrations were raised to 0.5 M (approach 1), 0.75 M (approach 2) and 1 M (approach 3 and 4). After 48 h, the concentrations of approach 3 and 4 were raised to 1.25 M. The final sample were taken after 115 h. To determine the ratio of crystallized to uncrystallized Lipase samples were taken after 25, 48 and 115 h and samples were centrifuged (10.000 rcf, 5 min). The total lipase activity in the supernatant represented the uncrystallized lipase whereas the total lipase activity in the redissolved pellets represented the crystallized lipase. Results are shown in FIGS. 13 and 14.

### REFERENCES

Northfield T C, McColl I. Postprandial concentrations of free and conjugated bile acids down the length of the normal human small intestine. Gut. 1973 July; 14(7):513-8
Koziolek M et al, Investigation of pH and Temperature Profiles in the GI Tract of Fasted Human Subjects Using the Intellicap System. J Pharm Sci. 2015 September; 104(9):2855-63
Brock et al., Novel ciliate lipases for enzyme replacement during exocrine pancreatic insufficiency. Eur J Gastroenterol Hepatol. 2016 November; 28(11):1305-12
Tondravi M M, Yao M C. Transformation of Tetrahymena thermophila by microinjection of ribosomal RNA genes. PNAS 1986 June; 83(12):4369-73
Gaertig J, Gorovsky M A. Efficient mass transformation of Tetrahymena thermophila by electroporation of conjugants. PNAS 1992 Oct. 1; 89(19):9196-200
Diogo M M, Silva S, Cabral J M, Queiroz J A. Hydrophobic interaction chromatography of Chromobacterium viscosum lipase J Chromatogr A. 1999 Jul. 23; 849(2):413-9.
Weide et al., A recombinase system facilitates cloning of expression cassettes in the ciliate Tetrahymena thermophila. BMC Microbiology 2007, 7:12
Weide et al., Secretion of functional human enzymes by Tetrahymena thermophila. BMC Biotechnol. 2006; 6: 19.
Chang V T. Structure. 2007 March; 15(3): 267-273
Hekmat, Bioprocess Biosyst Eng (2015) 38:1209-1231
Cassidy-Hanley, D. et al. (1997) Germline and somatic transformation of mating Tetrahymena thermophila by particle bombardment, Genetics, 146(1), pp. 135-147.
Gasteiger E. et al. (2005) Protein Identification and Analysis Tools on the ExPASy Server; (In) John M. Walker (ed): The Proteomics Protocols Handbook, Humana Press (2005). pp. 571-607
Jette J. F. et al (1994) Determination of lipase activity by a rhodamine-triglyceride-agarose assay, Anal Biochem 219(2), pp. 256-60

### Sequences

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones. Note that the respective SEQ ID NOs 1-7 refer to the enzymes with the signal peptides. SEQ ID NOS 8-14 (not shown in this list, yet in the electronic sequence listing) refer to the enzymes without the signal peptides. SEQ ID NOs 15-17 (not shown in this list, yet in the electronic sequence listing) refer to the protease enzymes without the propeptide.

| | | | UniProt |
|---|---|---|---|
| | Sequence (underlined: signal peptide, | | identifier/ |
| SEQ | italics: pro-peptide that is cleaved | | Gene Model |
| ID | off and is no longer ins the crystals)) | enzyme | identifier |
| 1 | MKLQLLLLVCLSFAACQSFTYTQSLAQDLAGFSLASYCNPK | Lipase | Q237S4 |
| | SIEQWNCGCACDKNPQGLRNVTILFNSTLQASGYLGYSTHH | 120 | TTHERM_00320120 |
| | DAIVVVFRGTVPWLIENWIADLNTFKTQYPLCQNCYVHQGF | | |
| | YNQFKQLKSQLVTSFTSLRQLYPNAKVFVTGHSLGAAMSAH | | |
| | SIPVIYQLNGNKPIDAFYNYGCPRVGDQTYANWFNSQNFAL | | |
| | EYGRINNAADPVPHLPPLLYPFSFFHYNHEIFYPSFVLFGN | | |
| | QHNQCQNAETIFGADGVIIAANVLDHLTYFGWDWSGSILTC | | |
| | Q | | |
| 2 | MKSIFLLIISLLLASCSQFQYNETLAQDLAGFSLASYCNPK | Lipase | Q237S3 |
| | YLQQWNCGSACKKNPNGLTDFSYLYNKTLKASGYIGYSAHH | 120 | TTHERM_00320130 |
| | DAIIVVFRGTVPWLIQNWIADLNTIKIQYPFCENCYVHKGF | | |
| | YKQFNQLKSQLIQSFTEIRQKYPSSKIFVTGHSLGAAMSFH | | |
| | SMPIIFELNGNKPIDAFYNYGSPRVGNEAYATWFNLONFAL | | |
| | QYGRINNAADPVPHLPPILFPFQFYHTNHEIFYTSFIEDGN | | |
| | KYEQCLDAEHKLCANSKIIAASVRDHLSYFGWNWATSILTC | | |
| | Q | | |
| 3 | MNKLQVLFIAAIVCTIGSTVYLLNKSSSDVQESQLTFPYDE | Lipase | Q237R4 |
| | NLAENLAGFSMASYCKASKIENWNCGASCKKNPEGLQDVYI | 0230 | TTHERM_00320230 |
| | MKNKTMNAAGFLAYSPAHDAIVVVFRGTVPWLIKNWISDIN | | |
| | TVKTKYSRCEKCYVHLGFFNAFKELQDQILTEFPKLKAKYP | | |
| | YSKNINYSKLNRRHSLGAAMSTHAVPVIYELNGNKPIDAFY | | |
| | NFGSPRVGDENYHQWFDSQNFTLQYGRINHRADPVPHLPPN | | |
| | YSPFTFTHIDHEVFYQTFKKPYTQCIETESLECADGIKIPL | | |
| | DIPDHLSYFGWDWATDILACQ | | |
| 4 | MSKLQFVLIAALLLVAVSAKTAQEWKTRNIYQIITDRFALG | Amylase | 17LW22 |
| | DGSKPYCDLNKQPYYCGGNFKGIENNLDYIQGMGFNAIWIS | 610 | TTHERM_00411610 |
| | PVPDNYPNQFHGYAAKNMTEINSHFGGADGLKSLIDACHKR | | |
| | DIWVMIDVVANHMGNTDQNYSENIPFNSPDHYHSYCIISDS | | |
| | DFATKNMYNIQHCRLAGLADLNQENSYVSNYLVNWIKDLVQ | | |
| | TYNVDGIRIDTVPEVHPDFWKQYTTSAGVFATGEVFDGDFG | | |
| | YVASYIPSVGSVLNYPWYFNMRDIFVNQKDMWGVRTYYQNW | | |
| | ANQHADLSILTPFVDNHDNPRFLSDQVENNGKDRNTRIQLL | | |
| | KGYTTFTLTSIGIPILYYGTEQYFSGNTDPYDREPLWNSLN | | |
| | TNSDMYKYVQSILYAKNITNAGLQGQTEAYCDQDLYAFFRG | | |
| | KLFVGVTSKFNQVTRQIVTHPFSNGEVICNIFYPTQDCLTI | | |
| | TNNSFNLVLIGGEAKIFVPKSLLQKSSQFLRSASS | | |
| 5 | MSQKITVTLVAIAAIAAITAAGIYYQNHQASQLEKSFKRNT | Protease | 17MB12 |
| | ILEQWNEFKQKFGKKYADQEFERYRIGVFAQNLEVIKNDPS | CCP1 | TTHERM_00683060 |
| | FGVTKFMDMTPQEFEQSYLSLQLQQNFNAEKVDGDFNGDID | | |
| | WTQKGAVTPVKDQGSCGSCWAFSAIGAVESALILNGEDKNI | | |
| | NLAEQELVDCATTPKYENEGCNGGWMDSAFDYIIDEKISQT | | |
| | KDYKYTARDGKCKDTSSFEKKSISGYKDIPQGDCKSLLNAL | | |
| | SQQPVAIAVDASSWQFYNKGVLSSCGSRLNHGVLLTGYVNE | | |
| | TYKVKNSWGTSWGEKGFIQLKSGNSCGLCNAASYPLA | | |
| 6 | MNKTLILALVGVLALTATTLVAFHNHSHNIRVDQDPATLFK | Protease | 17MB11 |
| | QFKQTYNKKYADPTFETYRFGVFTQNLEIVKTDSTFGVTQF | CCP2 | TTHERM_00683010 |
| | MDLTPAEFAQQFLTLHEKVNSTEVYRAQGEATEVDWTAKGK | | |
| | VTPVKNQGSCGSCWAFSTIGAVESALLIAGQGEQNTLNLAE | | |
| | QELVDCAKSPKYDSEGCNGGWMVEGFKYIIDNKISQTANYP | | |
| | YTAKDGKCKDTSSFKKFSISKYAEIPQGDCNSLNSALEQGP | | |
| | ISVAVDATNFQFYTSGVFKNCKANLNHGVLLVANVDSSLKI | | |
| | KNSWGPSWGEKGFIRLAAGNTCGVCNAASYPIV | | |
| 7 | MRTQLLIAAALGLTLLGLTSYLFLHKSTQVGYTDDQINMWK | Protease | 17MCX6 |
| | GFKKTYNKKFSSEDADQEAYRMNVFFDNVEYASQDSTMGIT | CCP5 | TTHERM_00530660 |
| | KFMDLTPVEFAQLYLNPIENVEGSIETFQAIQANGDIVVDW | | |
| | VAKGAVTPVKDQGGCGGCWSFATTGGVEGANFVYKNVLPNL | | |
| | SEQQLIDCDTQNSGCGGGLRDVALNYVKATGLATEQDYPYE | | |
| | AKDGKCRLEGKSHPWTVSGYTSIKQCADLVTAIQKAPVTVG | | |
| | IDASNLQFYTGGIFSKCATNINHGVLLVGYDSVNQSWKVKN | | |
| | SWGPNFGEHGYFQLSAKVTGDQIANTCGICSRAYAPYI | | |

What is claimed is:

1. A method for purifying proteins, which proteins are produced by homologous or heterologous expression in a ciliate host, from harvested cell culture fluid (HCCF) which was not chromatographically purified,
the method comprising the steps of
a) incubating a harvested cell culture fluid with a kosmotropic agent,
b) efflorescing protein by formation of crystals, and
c) optionally, harvesting effloresced protein,
wherein the method does not comprise a step of crosslinking the proteins before or after efflorescing.

2. The method according to claim 1, wherein the protein is a glycosylated protein.

3. The method according to claim 1 or 2, wherein the kosmotropic agent is at least one of
ammonium sulfate,
sodium dihydrogen phosphate, and/or
polyethylene glycol

4. The method according to any one of the aforementioned claims, wherein the harvested cell culture fluid comprises between ≥3 and ≤200 g/l of protein.

5. The method according to any one of the aforementioned claims, wherein for efflorescing the protein,
a pH of between ≥5 and ≤8 is established in the medium, and/or
a temperature of between ≥10 and ≤40° C. is established.

6. The method according to any one of the aforementioned claims, wherein, for the incubation of the harvested cell culture fluid, a kosmotropic salt concentration is established in the range of between ≥50 and ≤2500 mM.

7. The method according to any one of the aforementioned claims, wherein, for the incubation of the harvested cell culture fluid, a polyalcohol concentration is established in the range of between ≥2 and ≤25% w/v.

8. The method according to any one of the aforementioned claims, wherein the incubation of the harvested cell culture fluid with the kosmotropic agent is carried out over a period of between ≥10 min and ≤36 hrs.

9. The method according to any one of the aforementioned claims, wherein a pre-harvest cell culture fluid is subjected to one or more filtration and/or centrifugation steps prior to step a), to obtain the harvested cell culture fluid.

10. The method according to any one of the aforementioned claims, wherein the ciliate host is Tetrahymena sp.

11. The method according to any one of the aforementioned claims, wherein the homologous or heterologous expression is under control of
a metallothionein gene (MTT1) inducible promoter, and/or
a heat inducible promoter.

12. The method according to any one of the aforementioned claims, wherein the protein is an enzyme.

13. The method according to claim 12 wherein the protein is at least one selected from the group consisting of:
lipase
protease
amylase
glutenase
glutamine-specific cysteine protease
prolyl endopeptidases
saccharase, and/or
lactase.

14. The method according to any one of the aforementioned claims, wherein the enzyme is
a) Tetrahymena lipase according to any one of SEQ ID NOs 1-3 or 8-10,
b) Tetrahymena amylase according to SEQ ID NOs 4 or 11,
c) Tetrahymena protease according to any of SEQ ID NOs 5-7, 12-14 or 15-17, or
d) a variant of any one of the above enzymes having
(i) at least 90% amino acid sequence identity therewith, wherein said variant retains enzyme functionality, and/or
(ii) between 1 and 3 amino acid residues removed or added at the N-terminus and/or the C terminus

15. The method according to any one of claims 1-14, the method further comprising the step of solubilizing the effloresced protein.

16. A pharmaceutical intermediate which has been obtained with a method according to any one of claims 1-15.

17. The pharmaceutical intermediate according to claim 16, which comprises a solubilized form of the effloresced protein or the effloresced protein per se.

18. A purified protein that has been obtained with a method according to any one of claims 1-15.

19. A purified, crystallized protein or pharmaceutical intermediate which comprises a ciliate-type glycosylation pattern.

20. The intermediate according to any one of claim 16 or 17 or protein according to any one of claim 18 or 19, which is or comprises at least one of
a) Tetrahymena lipase according to any one of SEQ ID NOs 1-3 or 8-10,
b) Tetrahymena amylase according to SEQ ID NOs 4 or 11,
c) Tetrahymena protease according to any of SEQ ID NOs 5-7, 12-14 or 15-17, or
d) a variant of any one of the above enzymes having
(i) at least 90% amino acid sequence identity therewith, wherein said variant retains enzyme functionality, and/or
(ii) between 1 and 3 amino acid residues removed or added at the N-terminus and/or the C terminus

21. The intermediate according to any one of claim 16, 17 or 20 or the protein according to any one of claims 18-20, which is provided in crystalline form.

22. The intermediate according to any one of claim 16, 17, 20 or 21 or the protein according to any one of claims 18-21, which has a purity grade of ≥80%.

23. The intermediate according to any one of claims 16, 17, or 20-22 or the protein according to any one of claims 18-22, wherein the protein crystals have a length of between ≥2 and ≤1000 μM.

24. A pharmaceutic preparation comprising the intermediate according to any one of claims 16, 17, or 20-23 or the protein according to any one of claims 18-22, and optionally further one or more further pharmaceutically acceptable excipients.

25. The pharmaceutic preparation according to claim 24, wherein the protein is an enzyme.

26. The pharmaceutic preparation according to claim 25, wherein the enzyme is at least one selected from the group consisting of
lipase
protease
amylase
glutenase
glutamine-specific cysteine protease
prolyl endopeptidases
saccharase, and/or
lactase.

27. The pharmaceutic preparation according to claim 26, wherein the enzyme is or comprises
a) Tetrahymena lipase according to any one of SEQ ID NOs 1-3 or 8-10,
b) Tetrahymena amylase according to SEQ ID NOs 4 or 11,
c) Tetrahymena protease according to any of SEQ ID NOs 5-7, 12-14 or 15-17, or
d) a variant of any one of the above enzymes having
(i) at least 90% amino acid sequence identity therewith, wherein said variant retains enzyme functionality, and/or
(ii) between 1 and 3 amino acid residues removed or added at the N-terminus and/or the C terminus

28. The protein according to any one of claims 18-22, or the pharmaceutic preparation according to any one of claims 26-27 (for the manufacture of a medicament) for use in the treatment of a human or animal subject
being diagnosed for,
suffering from or
being at risk of developing
a disorder caused by enzyme deficiency or dysfunction, or for the prevention of such condition.

29. A method for treating or preventing a disorder caused by enzyme deficiency or dysfunction, which method comprises administration, to a human or animal subject, the enzyme according to any one of claims 18-22, or the pharmaceutic preparation according to any one of claims 26-27, in a therapeutically sufficient dose.

30. The protein for use according to claim 28 or the method according to claim 29, wherein the disorder caused by enzyme deficiency or dysfunction is at least one selected from the group consisting of
a lipid digestion deficiency and/or a digestive disorder
disorder caused by enzyme deficiency or dysfunction.
pancreatic exocrine insufficiency can have the following causes:
chronic pancreatitis
cystic fibrosis
main pancreatic duct obstruction
pancreatic resection
gastric resection
short bowel syndrome
hereditary hemochromatosis
celiac disease
Zollinger-Ellison syndrome
celiac sprue
sucrose intolerance or genetic sucrase-isomaltase deficiency (GSID), and/or
lactose intolerance

31. A dosage unit comprising the protein according to any one of claims 18-22, or the pharmaceutic preparation according to any one of claims 26-27.
